(19) United States
(12) Patent Application Publication
(10) Pub. No.: US 2006/0051365 A1
(43) Pub. Date: Mar. 9, 2006

(54) Immunogenic composition

(76) Inventors: Andre Francois Gorenflot, Montpellier (FR); Eric Precigout, Jacou (FR); Stephane Delbecq, Montpellier (FR); Theodorus Petrus Maria Schetters, Cuyk (NL)

Correspondence Address: INTERVET INC.;PATENT DEPARTMENT, PO BOX 318, MILLSBORO, DE, 19966-0318, US

(21) Appl. No.: 10/520,698

(22) PCT Filed: Sep. 7, 2003
(86) PCT No.: PCT/EP03/07477

§ 371 Date: Aug. 2, 2005

(30) Foreign Application Priority Data
Jul. 10, 2002 (EP) 02077800.7

(51) Int. Cl. A61K 39/005

(52) U.S. Cl. 424/191.1; 424/269.1

(57) ABSTRACT

The present invention provides an immunogenic composition comprising a fusion protein and a saponin adjuvant. The fusion protein comprises a heterologous hydrophobic peptide that is fused to the N-terminus and/or to the C-terminus of a core polypeptide. The core polypeptide comprises at least one protective epitope. The saponin adjuvant in this composition is present in a free form. Also provided are vaccines comprising the immunogenic composition and optionally an additional immunoactive component. Methods for the preparation of such vaccines, and use of the immunogenic composition for the manufacture of a vaccine are provided as well.

The present invention relates to an immunogenic composition comprising a fusion protein and a saponin adjuvant. Also to a vaccine, a method for the preparation of a vaccine, and use of an immunogenic composition.

In modern human and veterinary medicine, humans and animals will be vaccinated on a regular basis to prevent more or less debilitating diseases or infections, or to ensure a certain economic profitability. In 2000 the worldwide market value of biologicals for veterinary vaccinations alone was 2.5 billion USD (Wood-Mackenzie, 2001). The goal of any vaccination strategy is to obtain in a subject an activated status of the immune system that will protect against (symptoms of) infection or disease. To generate this immune protection, (part of) an infective agent is presented to the subject in a way that enables the immune system to prepare a protective response. Through the phenomenon of immunological memory, the subject's immune system is triggered to react more rapidly and strongly when the real infection, to which this vaccination was aimed, occurs in future.

To this purpose vaccines are commonly prepared from the targeted infective agent or a part thereof, such as by using live-, live-attenuated- or killed forms of an infective microorganism, or as protein subunit or nucleic acid thereof. Ideally all these will comprise (or encode) antigenic structures that induce the desired protective immune response in the human or animal target.

For reasons of safety, subunit vaccines are preferred. Subunits can then be produced by the cells of an expression system, via expression from a (recombinant) nucleic acid molecule.

Only when an antigen is capable of inducing an immune response that reduces signs of infection or disease it is called a protective antigen or an immunogen.

Consequently, in the field of subunit vaccines, an expressed protein may be antigenic, but is many times not found to be immunogenic. Let alone, to be sufficiently immunogenic for the production of a vaccine that is effective under field conditions as well as being economically feasible.

However, the optimization of a subunit's immunogenicity may negatively interfere with obtaining sufficient expression yield. This can be a result of the fact that overexpression may lead to an overload and collapse of the cellular apparatus for protein expression, or that the expression systems used for expression of heterologous proteins most times do not perform the same posttranslational modifications that would occur in the cells where the protein is naturally expressed. For instance, the processing of hydrophobic signal sequences that provide secretion or-surface exposure may cause problems in cells of an expression system through interactions with the cell's organelle- or outer membranes. It is therefore common practice to produce protein subunits in cellular expression systems without C-terminal hydrophobic anchoring signals, and to delete or modify N-terminal signal sequences. However the disadvantage of this approach is that this usually leads to low immunogenicity.

To overcome the problem of low immunogenicity of proteins produced in such a way, the addition to a subunit antigen of an adjuvant (an immune stimulatory substance) is commonly used. Such substances are relatively cheap and can be very efficient. Frequently used adjuvants are e.g. aluminum salts, oil emulsions, lipid A and saponins. However most of these substances cause some sort of local reaction in the form of tissue-irritation at the site of application. This causes discomfort to the subject and, in the case of veterinary applications, may result in a drop in productivity (e.g. milk or egg production, feed conversion) or condemnation of the meat or carcass at the slaughterhouse.

One of these adjuvants that have been found to be cytotoxic at effective concentrations is Quillaja saponin. The type and intensity of the local reactions to application of saponin are of course dependent of the quantity applied, and also of the purity of the material or particular batch that is being employed. B. Rönnberg et al., (1995, Vaccine, vol. 13, p. 1375-1382) describe hemolytic cytotoxicity for different fractions of Quillaja saponin. Toxic concentrations varied between 5 and 100 μg/ml for the different fractions. Local reactions were observed varying from swelling, to skin degeneration, and even death of the mice used in the experiments. In a similar study, Pillion et al. (1996, J. Pharm. Sci., vol. 85, p. 518-524), describe erythrocyte hemolysis by derivatives of Quillaja saponin in concentrations varying between 0.006 and 1.5 mM. Leung et al. (1997, BBA vol. 1325, p. 318-328) describe the cause of the cellular lysis to be the reaction of free saponin with the cholesterol in the cellular membranes.

The usual way of preventing the toxic properties of saponin to prevail over its adjuvant activity is by the incorporation in immune stimulatory complexes (ISCOMs) (WO 9611711). These are formed by mixing a saponin, a phospholipid and cholesterol. Under the right conditions particles with cage-like structures are formed. When an antigenic protein is integrated, particulate structures can be produced that present these antigens on the surface thereby mimicking the “natural” presentation of the antigens on an infected cell (reviewed in: Morein, B. & K. L. Bengtson, 1999, Methods, vol. 19, p. 94-102, and EP 109.942).

Alternatively ISCOM-matrix particles can be produced. These are ISCOM-like particles in which the subunit antigen is not integrated but is added later.

In both cases the cytotoxic effects of Quillaja saponin are neutralized. Hsu et al. (1996, Vaccine, vol. 14, p. 1159-1166) chose such an approach, by integrating a fusion peptide carrying an epitope into ISCOMs.

Unfortunately the generation of ISCOMs and ISCOM-matrixes is complex and costly, thereby making it uneconomical for instance for the general application in veterinary medicine. The use of saponin in free form, i.e. not integrated in an ISCOM is less complex and cheaper. However, at a concentration that it is normally effective as adjuvant, the disadvantageous cytoxicity of a saponin may manifest itself, as mentioned above.

It is an object of the current invention to provide for the first time an immunogenic composition of a protein antigen adjuvated with a saponin, which does provide an effective immune stimulation without significant adverse local effects, at cost effective production levels.

Surprisingly it was found now, that by fusing a hydrophobic peptide to the core of an immunogenic protein, this fusion protein could be combined with free saponin in such a low concentration that the resulting composition does not cause adverse local reactions, while still inducing an efficient immune response.

This is contrary to the common habit of incorporating saponin into ISCOMs or ISCOM-matrix particles for overcoming cytotoxicity. Also this counteracts the customary removal of hydrophobic amino acid (aa) stretches from subunit proteins expressed in a heterologous expression system. The possible loss in yield of fusion protein from the expression system is counterbalanced by the increased immunogenicity in the context of free saponin, and the reduction of adverse local reactions.

Consequently the current invention provides for the first time, a subunit vaccine adjuvated with saponin that is sufficiently safe, immunologically effective and has economic feasibility.

Therefore, in a first aspect the present invention provides an immunogenic composition comprising a fusion protein and a saponin adjuvant, characterized in that the fusion protein comprises a heterologous hydrophobic peptide which is fused to the N-terminus and/or to the C-terminus of a core polypeptide, the core polypeptide comprising at least one protective epitope, the saponin adjuvant being in a free form.

An mmunogenic composition is understood to be a composition that upon administration to a subject induces an immune response in that subject which reduces an infection or a disease. This implies stimulation of the components of the immune system. These can be the cellular components such as B— or T-lymphocytes, macrophages, killer cells, antigen presenting cells (APCs), etc., or the humoral components of the immune system, such as antibodies, cytokines (e.g. interferons or interleukins), etc.

For the purpose of the present invention the term “protein” refers to a molecular chain of amino acids. A protein is not of a specific length and can, if required, be modified in vivo or in vitro, by, for example, glycosylation, amidation, carboxylation or phosphorylation. Inter alia, peptides, oligopeptides and polypeptides are included within the definition. The protein or peptide can be of natural or synthetic origin.

A fusion protein is an assembly of two or more strands of amino acids that does not occur naturally. The strands can be of equal length, but usually they will differ in length, with the heterologous hydrophobic peptide(s) preferentially being shorter than the core polypeptide(s). The combination of the strands can be accomplished by several means, e.g.:
chemically, by coupling, conjugation or cross-linking, through dehydration, esterification, etc, of the amino acid sequences either directly or through an intermediate structure.
physically, by coupling through capture in or on a macromolecular structure by molecular biological fusion, through the combination of recombinant nucleic acid molecules which comprise fragments of nucleic acid capable of encoding each of the two, such that a single continuous expression product is finally produced.

A saponin is a surface-active glycoside that can be obtained from a plant by extraction. Well-known saponins are the Quillaja saponins that are extracted from the bark of the South American soap tree Quillaja saponaria [Molina] (Dalsgaard, K., 1974, Arch. Gesamte Virusforsch. vol. 44, p. 243-254). Depending on the method of extraction different saponin preparations will be obtained. Several preparations of Quillaja saponin are available commercially: Spikoside™ from Iscotec AB, Sweden, Quil A™ from Superfos AS, Denmark, Q-vac™ from Nor-Vet, Denmark, and QS-21™ from Antigenics, USA, and Vax-Sap™, from Desert King, Chili. Some are crude, while others are more purified preparations.

For the purpose of the present invention, a saponin is in a free form if it has not been purposively mixed with cholesterol and phospholipid to produce ISCOM or ISCOM-matrix particles.

An adjuvant in general is a substance that boosts the immune response of the receiving subject in a non-specific manner.

The core polypeptide that is to be connected with the hydrophobic peptide, relates to a polypeptide that does not contain the N— or C-terminal hydrophobic regions that are present in a naturally occurring form of that protein. Thus the core protein for the invention is a component of a protein from an agent or organism. Proteins that do not contain hydrophobic ends in their natural form can serve as “core” without further modifications to their native composition.

These hydrophobic regions can be cut off from the protein by using chemical or enzymatic procedures. Preferentially the nucleic acid sequence encoding such a protein is modified by genetic engineering techniques in such a way that these hydrophobic regions are no longer expressed.

In principle any protein of medical importance may serve as core polypeptide for the invention. Preferably the core polypeptide is a component of an infective agent or a biological factor that is known or expected to cause disease to humans or animals. For instance infective agents or factors causing cancer, HIV or AIDS, (auto-) immune disease, neurological-, neurodegenerative-, respiratory-, or dermal afflictions.

More preferably the core polypeptide is a component of proteins that have shown an immunogenic potency in vaccination studies, for instance after being isolated from the infective agent, after having been expressed in an expression system, through use as an insert in a live recombinant carrier microorganism (LRCM), or after use as DNA vaccine.

Examples of such proteins that could serve as core polypeptide for the invention are proteins from the envelope, matrix-, organelles, or nucleus or from non-structural- or glycoproteins from parasites, bacteria, or viruses, and the proteins they induce or interact with in their hosts.

Even more preferentially, the core polypeptide is a component of:
Parasitic proteins: for instance parasitic enzymes (soluble or internal) from Eimeria, or soluble protective antigens such as exoantigens or merozoite surface antigens from Babesia.
Viral proteins: e.g. proteins from retroviruses: envelope proteins gp20, gp40, gp120, rev, tat or nef proteins, retroviral group specific antigens; Newcastle disease virus fusion or hemagglufinin-neuraminidase protein; influenza viral hemagglutinin or neuraminidase proteins; Bimaviral VP2; Coronaviral spike or matrix proteins; Pestiviral envelope proteins E^{ms}, E1 or E2; porcine reproductive and respiratory disease virus viral proteins.
Bacterial proteins: such as toxins, adhesins, fiber-, fimbriae-, pilum- or outer-membrane proteins.
Induced proteins: such as interleukins; interferons; cancer-antigens such as from the p53 cascade, HER-2/Neu, Carcino-embryonic antigen; tumor-induced angiogenesis factors like fibronectin or gangliosides; receptor molecules.

The term heterologous for the purpose of the present invention refers to the origin of the hydrophobic peptide, in relation to the core polypeptide to which it is to be connected. Thus a peptide is of heterologous origin, if it is not part of the same protein from which the core polypeptide is a component in a particular species of organism or agent. For instance, the C-terminal hydrophobic peptide of the Bd37 protein. from Babesia bovis, would qualify as a heterologous peptide if it was to be fused to the core polypeptide of the Bd37 protein homologue from B. divergens.

In general a hydrophobic peptide is understood to be a strand of amino acids that have a preference for a non-polar environment. Such peptides are also known in the art as lypophylic or water-insoluble. Several computer algorithms have been developed that enable assessment of a peptide's hydrophobicity. For the purpose of the present invention, the hydropathy algorithm of Kyte & Doolittle (J. Kyte and R. F. Doolittle, 1982, J. Mol. Biol., vol. 157, p. 105-132) is used. This widely used program calculates a moving average of the free transfer energy over a certain window of amino acids, thereby producing a hydrophobicity profile that can be represented as a table of data points, or as a graph. In the table the moving average numbers that are positive indicate hydrophobic amino acids; in the graphical representation of the hydrophobicity profile, the part below the median is hydrophobic.

This algorithm is available in most software packages for protein analysis or structure prediction, and through the internet via the web-pages of most bioinformatics institutes.

For the present invention, a peptide is considered hydrophobic if 60% or more of the data points from the Kyte-Doolittle hydrophobicity analysis indicate a hydrophobic value; the hydrophobicity must be calculated using a window of 5 amino acids. The percentage hydrophobicity is calculated from the data table from such an analysis: the number of hydrophobic data points (the moving average numbers that are positive) is divided by the total number of aa of the peptide. Preferably 70% of the data points from the Kyte-Doolittle hydrophobicity analysis are hydrophobic, more preferably 80%, 85%, 90%, 95%, 98%, or 100%, in increasing order of preference.

To illustrate the Kyte-Doolittle hydrophobicity profile, Table 1 describes hydrophobic peptides from a variety of sources that can be used to produce a fusion protein for the invention, while their accompanying hydrophobicity profiles are represented in FIG. 1. This figure also lists the percentage of hydrophobicity.

A hydrophobic peptide for the present invention preferentially comprises a sequence of between 3 to 200 amino acids, more preferably 4 to 150, even more preferably 4 to 100, still even more preferably 5 to 75, and most preferably 6 to 50 amino acids.

The hydrophobic peptides that can be used to produce a fusion protein for the invention, can be derived from different regions of the donor protein they originate from. For instance:
N-terminally:
   N-terminal hydrophobic peptides such as signal sequences can be derived from proteins as diverse as melittin (honey bee venom), human tissue plasminogen activator (TPA), yeast mating pheromone alpha-factor, baculovirus envelope glycoprotein gp67, or pseudorabiesvirus gX. These signal sequences have been reviewed by Izard & Kendall (1994, Mol. Microbiol., vol. 13, p. 765-773), Claros et al. (1997, Curr. Opin. Struct Biol., vol. 7, p. 394-398), and Lammertyn & Anne (1998, FEMS Microblol. Lett., vol. 1, p. 1-10).
Internally:
   Internal hydrophobic peptide sequences are for instance transmembrane regions (TMRs), these can be situated close to the N— or C-terminus of a protein, but also further internal, for instance in the case of proteins with membrane spanning segments. Examples are the membrane anchor from-measles virus hemagglutinin-neuraminidase; transmembrane signaling receptors like the “seven membrane-spanning domain” receptors; membrane channels; cellular pores, and pumps, etc. Such signals are reviewed by Goder & Spiess (2001, FEBS Left., vol. 31, p. 87-93), von Heijne & Manoil (1990, Protein Eng., vol. 4, p. 109-112), and in general textbooks like Molecular Biology of the Cell, by Alberts et al. (2002, Garland Science publ., ISBN: 0815340729).
C-terminally:
   C-terminal hydrophobic peptides are for instance glycosyl phosphatidylinositol (GPI) anchoring signals, for instance from the C-terminus of the human CD14 (monocyte differentiation antigen (NCBI protein database accession number P08571), the chicken TGF-β neurotrophic factor receptor 1 (NCBI acc. nr. 013156), and the Sacharomyces cerevisiae cell wall protein 1 (see Table 1 and FIG. 1). The characteristics of such GPI anchors are reviewed by P. Englund (1993, Ann. Rev. Biochem., vol. 62, p.121-138), and Chatterjee & Mayor (2001, Cell. Mol. Life Sci., vol. 58, p. 1969-1987).

NB: The NCBI protein- and nucleic acid sequence databases can be reached through the internet at: http://www.ncbi.nim.nih.gov, and their use for the purpose of obtaining a. o. nucleic acid- or protein sequences is well-known in the art.

TABLE 1

Examples of hydrophobic peptides
for use in the invention

Peptide's

| | | | |
|---|---|---|---|
| Donor | NCBI | location | Peptide's aa sequence |

| | | | | |
|---|---|---|---|---|
| protein | acc. nr. | aa nr. | in donor | (from N- to C-terminus) |

| | | | | |
|---|---|---|---|---|
| Melittin | MK92098 | 1-21 | N-term. | MKFLVNVALVFMVVYISYIYA |
| DAF | B26359 | 352-381 | C-term. | TSGTTRLLSGHTCFTLTGLLGT |
| | | | | LVTMGLLT |
| CWP 1 | BAA07193 | 219-239 | C-term. | GAKAAVGMGAGALAVAAAYLL |
| MV HN | P35971 | 35-58 | Internal | PYVLLAVLFVMFLSLIGLLAIAGI |
| HHV-4 | S27922 | 281-300 | Internal | EENLLDFVRF MGVMSSCNSS |
| EBNA-3C | | | | |

DAF = Decay accelerating factor (CD 55); CWP 1 = Sacharomyces cell wall protein 1; MV HN = measles virus hemagglutinin-neuraminidase; HHV-4 EBNA-3C = human herpesvirus 4, nuclear antigen EBNA-3C.

The computer package used to calculate the hydrophobicity profiles represented in FIG. 1 (Clone Manager, SciEd software, Durham, USA) attributes a value above zero to hydrophobic amino acid stretches, while hydrophilic stretches score negative values. The moving average is calculated over a window of 5 aa.

It goes without saying that when employing different computer packages to calculate such a profile, there may be slight differences. However, the difference between hydrophobic and hydrophilic amino add regions will remain clear.

In literature, fusion proteins are commonly expressed to facilitate purification during downstream processing. However, the fusion peptides used for that purpose do not qualify as hydrophobic peptides for the invention.

An epitope is understood to be that part of an antigenic molecule to which a T-cell receptor will respond, or to which B-cells will produce antibodies. A protective epitope for the invention will therefore induce specific T-cells or activate B-cells to produce specific antibodies such that these cells or antibodies give rise to an immune reaction that interferes with the course of an infection or disease. Thus, through such protective epitopes, a protective immune response can be generated.

The protective epitope is comprised in the core polypeptide part of the fusion protein for the invention.

The heterologous hydrophobic peptide that is connected to the core polypeptide may also contain an epitope. The presence of more than one epitope in the fusion protein may even enhance the immunologic effectivity of the fusion protein of the present invention.

Today, a variety of techniques are available to easily identify protein epitopes. One empirical method that is especially suitable for the detection of B-cell epitopes, is the so-called PEPSCAN method. This is described by Geysen et al. in Proc. Natl. Acad. Sci. USA, vol. 81, p. 3998-4002 (1984), J. Imm. Meth. vol. 102, p. 259-274 (1987), and patent applications WO 84/03564 and WO 86/06487, and U.S. Pat. No. 4,833,092. The PEPSCAN method is an easy to perform, quick and well-established method for the detection of epitopes. It comprises the synthesis of a series of peptide fragments progressively overlapping the protein under study, and subsequent testing of these polypeptides with specific antibodies to the protein.

Also, given the amino acid (or nucleic acid) sequence of any protein (or gene encoding it), computer algorithms are available that can designate specific protein regions as the immunologically important epitopes on the basis of their sequential and/or structural agreement with epitopes that are now known. The determination of these regions is based on a combination of the hydrophilicity criteria according to Hopp and Woods (Hopp T. P and Woods, K R., 1981, Proc. Natl. Acad. Sci. U.S.A., vol. 78, p. 3824-3828), and the secondary structure aspects according to Chou and Fasman (Adv. in Enzymology, vol. 47, p. 45-148 (1987), and U.S. Pat. No. 4,554,101). An example of a program employing a combination of such algorithms is PepPlot (Gribskov et al., 1986, Nucl. Acids Res. vol. 14, p. 327-334).

Because T-cell epitopes are normally hidden in hydrophobic regions of a protein that fold away from the polar, hydrophilic exterior (which is generally the location of B-cell epitopes), the hydrophobic character of the heterologous peptide to be fused to the core polypeptide is particularly suited for incorporating a T cell epitope.

As is reviewed by Berzofsky et al. (1987, Immunol. Rev., vol. 98, p. 9-52), T-cell epitopes consist of short linear stretches of amino acids, and can only be presented to the immune system in the context of MHC-I after their processing by APCs.

One of many examples is the EBNA-3C nuclear antigen from human herpes virus 4 (NCBI acc. nr. S27922), also described in Table 1 and in FIG. 1.

T-cell epitopes can be predicted from a sequence by computer like B-cell epitopes, with the aid of Berzofsky's amphiphilicity criterion (1987, Science, vol. 235, p.1059-1062). This was reviewed by Lu et al. (1992, Vaccine vol. 10, p. 3-7). An illustration of the effectiveness of using these methods was published by H. Margalit et al. (1987, J. of Immunol., vol. 138, p. 2213-2229) who describe success rates of 75% in the prediction of T-cell epitopes using such methods.

The heterologous hydrophobic peptide and/or the core polypeptide may also contain other immune-activating signatures. Such signatures may comprise immuno-stimulatory signals like from chemokines or immunotoxins.

The preferred way to produce a fusion protein for the invention is by using genetic engineering techniques and recombinant expression systems. These may comprise using (recombinant) nucleic acid sequences, LRCMs and host cells.

Nucleic acid sequences that can be used to encode a fusion protein for the invention can be obtained, manipulated and expressed by standard molecular biology techniques that are well-known to the skilled artisan, and are explained in great detail in standard text-books like Sambrook & Russell: Molecular cloning: a laboratory manual (2000, Cold Spring Harbor Laboratory Press; ISBN: 0879695773).

To construct a nucleic acid encoding a fusion protein for the invention, preferably DNA plasmids are employed. Such plasmids are useful e.g. for enhancing the amount of DNA-insert, for use as a probe, and as tool for further manipulations. Examples of such plasmids for cloning are plasmids of the pBR, pUC, and pGEM series, all these are available from several commercial suppliers.

The DNA encoding the polypeptide core and the hydrophobic peptide can e.g. be cloned into separate plasmids, be modified to obtain the desired conformation and next be combined into one recombinant plasmid; the reading frame of peptide and core is aligned in such a way that a single continuous fusion protein can be expressed.

Modifications to the nucleic acid sequences that could encode the hydrophobic peptide and/or the core polypeptide for the invention may be performed e.g. by using restriction enzyme digestion, by site directed mutations, or by polymerase chain reaction (PCR) techniques. Standard techniques and protocols for performing PCR are for instance extensively described in C. Dieffenbach & G. Dveksler; PCR primers: a laboratory manual (1995, CSHL Press, ISBN 879694473).

For the purpose of protein purification, detection, or improvement of expression level, additional sequences may be added. This may result in the final insert in the recombinant nucleic add molecule or plasmid being larger than the sequences coding for the fusion of the hydrophobic peptide and the core polypeptide. When such additional elements are inserted in frame, these become an integral part of the fusion protein for the invention.

An essential requirement for the expression of a nucleic add sequence from a recombinant nucleic add molecule is that the nucleic acid is operably linked to a transcriptional regulatory sequence such that it is capable of controlling the transcription of the nucleic add sequence. Transcriptional regulatory sequences are well-known in the art and comprise i.a. promoters and enhancers. It is obvious to those skilled in the art that the choice of a promoter extends to any eukaryotic, procaryotic or viral promoter capable of directing gene transcription, provided that the promoter is functional in the expression system used.

Bacterial, yeast, fungal, insect, and vertebrate cell expression systems are used very frequently. Such expression systems are well-known in the art and generally available, e.g. commercially through Invitrogen (the Netherlands).

A host cell to be used for expression of a fusion protein for the invention may be a cell of bacterial origin, e.g. from Escherichia coli, Bacillus subtilis, Lactobacilus sp. or Caulobacter crescentus, in combination with the use of bacteria-derived plasmids or bacteriophages for expressing the sequence encoding the fusion protein. The host cell may also be of eukaryotic origin, e.g. yeast-cells in combination with yeast-specific vector molecules, or higher eukaryotic cells, like insect cells (Luckow et al; Bio-technology 6: 47-55 (1988)) in combination with vectors or recombinant baculoviruses; plant cells in combination with e.g. Ti-plasmid based vectors or plant viral vectors (Barton, K. A. et al; Cell vol. 32, p. 1033 (1983)); or mammalian cells like Hela cells, Chinese Hamster Ovary cells (CHO) or Crandell-Rees feline kidney-cells, also with appropriate vectors or recombinant viruses.

An example of an expressed fusion protein for the invention, is the avian influenza virus H5 HA protein-core (e.g. derivable from NCBI acc. nr. CAC28131), fused to a heterologous hydrophobic sequence, and expressed in the baculovirus expression vector system, see example 4.

Next to these expression systems, plant cell, or parasite-based expression systems are attractive expression systems. Parasite expression systems are e.g. described in the French Patent Application, publication number 2 714 074, and in US NTIS publication no. U.S. Ser. No. 08/043109 (Hoffman, S. and Rogers, W., 1993). Plant cell expression systems for polypeptides for biological application are e.g. discussed in R. Fischer et al. (1999, Eur. J. of Biochem., vol. 262, p. 810-816), and J. Larrick et al. (2001, Biomol. Engin. vol. 18, p. 87-94).

Expression may also be performed in so-called cell-free expression systems. Such systems comprise all essential factors for expression from an appropriate recombinant nucleic acid, operably linked to a promoter that will function in that particular system. Examples are the E. coli lysate system (Roche, Basel, Switzerland), or the rabbit reticulocyte lysate system (Promega corp., Madison, USA).

In a preferred form of this aspect of the invention, the immunogenic composition according to the invention is characterized in that the core polypeptide is a component of a protein of an organism of the phylum Apicomplexa.

To the phylum Apicomplexa belong several taxonomic groups with members of (veterinary) medical relevance, e.g. the Piroplasmida, the Coccidia, and Hemosporida. The invention could for instance very well be used to produce a fusion protein comprising a Plasmodium yoelli MSP-1₁₉ core (Ling et al., 1994, Parasite Immunol., vol. 16, p. 63-67). that is fused to a heterologous hydrophobic peptide.

In a more preferred form, the immunogenic composition according to the invention is characterized in that the core polypeptide is a component of a protein of an organism of the Piroplasmida or of the class Coccidia.

To the Piroplasmida belong several relevant taxonomic groups, e.g. the Babesiidae and the Theileriidae, with for example respective relevant genera Babesia and Theileria.

To the Coccidia belong a.o. the Eimeriidae, Cryptosporidiidae and the Sarcocystidae, comprising relevant genera such as Eimeria, Cryptosporidium, Neospora and Toxoplasma.

In an even more preferred form of this aspect, the immunogenic composition according to the invention is characterized in that the core polypeptide is a component of a protein of an organism of the genera Eimeria or Babesia.

As outlined above, the hydrophobic peptides that can be used to produce a fusion protein for the invention, can be derived from different regions of the donor protein they originate from. For instance: N-terminally, internally, or C-terminally.

Therefore, in an alternate preferred embodiment of the first aspect of the invention, the heterologous hydrophobic peptide is from an N-terminal hydrophobic sequence.

In an other alternate preferred embodiment of the first aspect of the invention, the heterologous hydrophobic peptide is from an internal hydrophobic sequence.

In yet an other alternate preferred embodiment of the first aspect of the invention, the heterologous hydrophobic peptide is from a C-terminal hydrophobic sequence.

In a preferred embodiment the C-terminal hydrophobic sequence is from decay accelerating factor (DAF).

Decay accelerating factor, also known as CD 55, has a hydrophobic amino acid region at its C-terminus. This functions as a GPI anchor (reviewed a. o. by Nicholson-Weller & Wang, 1994, J. Lab. Clin. Med., vol. 123, p. 485-491). The hydrophobicity profile is illustrated in FIG. 1. As represented in Table 1, the amino acid sequence of the DAF C-terminus that was used corresponds to amino acids Thr-352 up to and including Thr-381, which is the last aa of the peptide sequence (NCBI acc. nr: B26359).

The use of the DAF C-terminal hydrophobic region in fusion protein constructs has been described before (e.g. Field et al., 1994, J. Biol. Chem. vol. 8, p. 10830-10837). However this was always directed to the purpose of studying the mechanism of anchoring and release of surface proteins.

An illustration of cloning and expression of a fusion protein for the invention is e.g. the fusion of the human DAF C-terminus to the B. divergens Bd37 core polypeptide, as described in examples 1 and 2. Vaccination with an immunogenic composition based on this fusion protein is described in example 3.

In still another preferred embodiment of the first aspect of the invention, the saponin adjuvant is Quillaja saponin.

Saponins have been extensively described above.

In the most preferred embodiment of the first aspect of the invention, the immunogenic composition according to the invention is characterized in that the fusion protein comprises the Babesia divergens Bd37 core polypeptide with C-terminal fusion of the C-terminal hydrophobic sequence from DAF, and the saponin adjuvant is Quil A.

The parasite B. divergens is transferred via an arthropod host, causes babesiosis In bovines, and is a known zoonosis for humans. This was reviewed by Kuffler, K. L. (“Babesiosis of domestic animals and man”, M. Ristic ed., 1988, CRC Press, Inc., Boca Raton, Fla., USA.

The B. divergens, isolate Rouen 1987, was derived from a human Babesiosis patient, and was used to study the Bd37 exoantigen, as described by B. Carcy et al. (1995, Infect. & Immun., vol. 63, p. 811-817). The nucleotide sequence of the corresponding cDNA is available under acc. nr. AJ422214 from the NCBI database. The core polypeptide of Bd37 that can be used as a core polypeptide for the invention, comprises the Bd37 sequence, available protein sequence under NCBI acc. nr CAD19563, without the N-terminal and C-terminal hydrophobic sequences. For instance the Bd37-core consists of Ser-25 up to and including Ser-316 from NCBI acc. nr: CAD19563.

Publications exist that describe vaccination experiments with a Bd37 core polypeptide: N. Grande et al. (1998, Parasitology Int., vol. 47, p. 269-279) performed vaccination experiments with B. divergens exoantigens (amongst others containing the soluble version of Bd37 which is similar to the core polypeptide) in free Quil A. However, no fusion protein for the invention was used or contemplated.

Another aspect of the invention relates to an immunogenic composition for use in a vaccine.

A further aspect of the present invention relates to a vaccine characterized in that it comprises an immunogenic composition according to the invention and a pharmaceutically acceptable carrier.

A pharmaceutically acceptable carrier is understood to be a compound that does not adversely effect the health of the subject to be vaccinated, at least not to the extent that the adverse effect is worse than the effects seen when the subject is not vaccinated.

A pharmaceutically acceptable carrier can be e.g. sterile water or a sterile physiological salt solution. In a more complex form the carrier can e.g. be a buffer.

The vaccine, or the vaccine with additional immunoactive component(s) according to the invention may additionally comprise a so-called “vehide”. A vehicle is a compound to which the fusion protein adheres, without being covalently bound to it. Such vehicles are i.a. bio-microcapsules, micro-alginates, liposomes and macrosols, all known in the art. In addition, the vaccine may comprise one or more suitable surface-active compounds or emulsifiers, e.g. Span™ or Tween™.

Often, a vaccine is mixed with stabilizers, e.g. to protect degradation-prone proteins from being degraded, to enhance the shelf life of the vaccine, or to improve freeze-drying efficiency. Useful stabilizers are i.a. SPGA (Bovarnik et al., 1950, J. Bacteriology, vol. 59, p. 509 ), carbohydrates e.g. sorbitol, mannitol, trehalose, starch, sucrose, dextran or glucose, proteins such as albumin or casein or degradation products thereof, and buffers, such as alkali metal phosphates. In addition, the vaccine may prior to application be suspended in a physiologically acceptable diluent. It goes without saying, that other ways of adjuvating, adding vehicle compounds or diluents, emulsifying or stabilizing a protein are also embodied in the present invention.

A preferred embodiment of the vaccine according to the invention relates to a vaccine characterized in that it comprises at least one additional immunoactive component.

The additional immunoactive component(s) may be an antigen, an immune enhancing substance, and/or a vaccine; either of these may comprise an adjuvant.

The additional immunoactive component(s) when in the form of an antigen may consist of any antigenic entity of human or veterinary importance. It may for instance comprise a biological or synthetic molecule such as a protein, a carbohydrate, a lipopolysacharide, a nucleic acid encoding a proteinacious antigen, or a recombinant nucleic acid molecule containing such a nucleic acid operably linked to a transcriptional regulatory sequence. Also a host cell comprising such a nucleic add, recombinant nucleic acid molecule, or LRCM containing such a nucleic add, may be a way to deliver the nucleic acid or the additional antigen. Alternatively it may comprise a fractionated or killed microorganism such as a parasite, bacterium or virus.

The additional immunoactive component(s) in the form of an immune enhancing substance may e.g. comprise chemokines, and/or immunostimulatory sequences (e.g. CpG motifs).

Alternatively, the immunogenic composition, or the vaccine according to the invention, may itselve be added to a vaccine.

The vaccine according to the invention can be administered to a subject according to methods known in the art, depending on the particular disease to be protected against.

Such methods comprise application e.g. parenterally, such as through all routes of injection into or through the skin: e.g. intramuscular, intravenous, intraperitoneal, intradermal, submucosal, or subcutaneous. Also, they may be applied by topical application as a drop, spray, gel or ointment to the mucosal epithelium of the eye, nose, mouth, anus, or vagina, or onto the epidermis of the outer skin at any part of the body. Other possible routes of application are by spray, aerosol, or powder application through inhalation via the respiratory tract. In this last case the particle size that is used will determine how deep the particles will penetrate into the respiratory tract. Alternatively, application can be via the alimentary route, by-combining with the food, feed or drinking water e.g. as a powder, a liquid, or tablet, or by administration directly into the mouth as a liquid, a gel, a tablet, or a capsule, or to the anus as a suppository.

It goes without saying that the optimal route of application will depend on the particularities of the infection or disease that is to be prevented or ameliorated, and the characteristics of the immunogenic composition or vaccine that is used.

Target subjects for the vaccine according to the invention may be humans or animals; animals may be fish, amphibians, reptiles, birds or mammals. These targets may be healthy or diseased, and may be seropositive or -negative. The target subjects can be of any age at which they are susceptible to the vaccination and/or to the infection or disease it aims to protect against.

Vaccines based upon a fusion protein for the invention can very suitably be administered in amounts containing between 0.1 and 100 micrograms of protein per subject, smaller or larger doses can in principle be used.

The adverse effect that may be observed at the site where a vaccine or a pharmaceutical compound has been applied to a subject, is commonly known in the art as the “local” or “adverse” reaction, and may be observed and scored in various ways.
observationally: discomfort or lethargy in the subject, temperature increase, locomotory disfunction, reduced feeding, drop in production of e.g. milk, eggs, or feed-conversion rate.
macroscopically: size of a swelling, color, presence of hemorrhage or edema, tissue consistency, abscess formation or necrosis.
microscopically: localization of lesions to particular tissue- or cell-types, type of lesions, severity etc.

It is one of the merits of the present invention that saponin adjuvant can be used in free form in such a low concentration that there is no significant development of signs of such local reactions.

For the purpose of the invention, saponin concentrations between 1 μg and 5 mg per dose can be used depending on the specific composition and target species.

Preferably saponin concentrations are employed such that saponin micelles are not formed intentionally. For instance for Quil A this would mean using a concentration below the critical micelle concentration (cmc) of 300 μg/ml (0.03%) (Morein, B. et al., 1984, Nature, vol. 308, p. 457-460), and for QS-21 a concentration below the cmc of 26 μM (C. R. Kensil, Chapter 15, in: “Vaccine adjuvants”, D. T. O'Hagan ed., Humana press 2000, ISBN: 0896037355). The concept of micelle forming at cmc is well-known to man skilled in the art, and is e.g. described in Remington: “The science and practice of pharmacy” (chapter 21, 20^{th} ed. 2000, Lippincot, USA, ISBN: 683306472).

Vaccines based upon the immunogenic composition according to the invention are also very suitable as marker vaccines

For reasons of e.g. stability or economy the vaccine of the invention, or the vaccine with additional immunoactive component(s) may be freeze-dried. Generally this will enable prolonged storage at temperatures above zero° Celsius.

Procedures for freeze-drying are known to persons skilled in the art; equipment for freeze-drying at any scale is available commercially.

Therefore, in a more preferred embodiment the vaccine according to the invention is characterized in that it is in a freeze-dried form.

An other aspect of the present invention is a method for the preparation of a vaccine according to the invention, characterized in that the method comprises admixing an immunogenic composition according to the invention and a pharmaceutically acceptable carrier.

The immunogenic composition and the pharmaceutically acceptable carrier can be combined into a vaccine in several ways, e.g. via admixing. The resulting vaccine can be in several forms, e.g.: a liquid, a gel, an ointment, a powder, a tablet, or a capsule, depending on the desired method of application to the target.

An other aspect of the invention comprises the use of an immunogenic composition according to the invention for the manufacture of a vaccine.

The present invention will now be further described with reference to the following, non-limiting, examples.

### EXAMPLES

### Example 1

### Cloning of Recombinant Constructs

Bd37 cDNA:

The isolation and cloning of the Bd37 gene from the parasite B. divergens, strain Rouen 1987, has been extensively described in EP 1050541, example 1. In brief: a cDNA expression-library was prepared from the mRNA of B. divergens, strain Rouen 1987 infected human erythrocytes. The library was screened with an anti-Bd37 polyclonal antiserum. From a positive clone the insert was rescued, and subcloned to generate plasmid pBK-CMV-Bd37.

His+Bd37-Core

Using the pBK-CMV-Bd37 plasmid (EP 1050541, example 1) as template, the central part of the gene of Bd37 without N— or C-terminal hydrophobic sequences the Bd37 insert was amplified using primers pQEUp and pQEDown (see Table 2) by 20 cycles of 1 min 94° C., 1 min 55° C., and 1 min 72° C., with 200 μM of each dNTP, 200 nM of each primer, 2.5 U TurboPfu™ polymerase enzyme (Stratagene), in 50 μl final volume. Template DNA was used in quantities between 50 ng and 1 μg depending on the desired yield.

Primer pQEUp creates an in frame BamHI site, while primer pQEDown creates a HindIII site. After BamHI-HindIII digestion a nucleic acid was obtained, comprising the part coding for the core of the Bd37 protein from Ser-25 up to and including Ser-316 (from NCBI acc. nr: CAD19563).

Primers were synthesized by Sigma-Genosys (Cambridge, UK).

The PCR product was purified by agarose gel electrophoresis, by loading onto a 0.8% agarose gel (electrophoresis grade, Eurobio, France) running in 0.5× TAE (made from 25× TAE stock solution, Euromedex) at 100V in a run-One™ electrophoresis system (Bioblock, France). The band corresponding to the desired product was excised from the gel, and the DNA was isolated from the gel slices using a gel-extraction Spin kit™ (Q-Bio-Gene), the DNA fragment was digested with BamHI and HindIII and gel purified again.

The resulting fragment was ligated into BamHI-HindIII digested pQE-30 vector (Qiagen), by ligation with T4 DNA ligase (MBI Fermentas, France) in 1× ligase buffer (MBI Fermentas) supplemented with 2 mM ATP (Sigma), at room temperature during 3 hours. The ratio vector:insert was usually 1:3, wherein the amount of digested vector used was between 0.5 and 1 μg.

The plasmid had been phosphatase-treated after the digestion, before the ligation with Calf Intestinal Alkaline Phosphatase (CIAP, Promega) in 1× CIAP buffer (Promega) during 1 h at 37° C.

The ligation mix was transformed into JM109 supercompetent™ E. coli cells (Promega). These cells were plated on ampicilin containing agar plates, and colonies were checked for expression of Bd37 protein by protein miniexpression. Briefly, a small scale (5 ml) bacterial culture in LB medium was initiated by 10-fold dilution of an overnight culture, after 2 h incubation at 37° C. with shaking, recombinant protein expression was induced by addition of 1 mM IPTG (Euromedex). After 3 h of induction, cells were harvested by centrifugation (15 min, 4000×g) and lysed in 1 ml of denaturing lysis buffer (8 M urea, 1% v/v Triton X-100, 50 mM Tris, pH=8). Lysates were sonicated for 2 minutes with 2 second pulse-pause cycles on ice, and centrifugated (15000×g, 10 min). Clarified lysates were incubated 20 min on ice with occasional shaking in presence of 50 μl of NiNTA agarose resin (Qiagen). Loaded resin was washed thrice with 1 ml of washing buffer (8 M urea, 1% v/v TX-100, 50 mM Tris, pH=6.3) and protein eluted with elution buffer (8 M urea, 1% v/v TX-100, 50 mM Tris, pH=4.5). The presence of recombinant protein was assessed by SDS-PAGE in 12% poly-acrylamide gel, which was stained with Coomassie Brilliant blue (CBB) and by Western blot with anti-Histag monoclonal antibody (Qiagen).

From one ampicilin-resistant colony, positive for Bd37 expression, a bacterial culture was produced by overnight incubation in 5 ml of LB medium, at 37° C. with shaking, and plasmid pQE-His-Bd37 was isolated using the JetQuick™ miniprep kit (Q-Bio-Gene, France) using 2 ml of the overnight culture.

Through the use of the pQE-30 vector a 6 aa Histidine linker is fused in frame to the Bd37 core polypeptide.

His+GST

Procedures similar to the ones for the 6× His-Bd37-core construct described above, were used to produce pQE-His-GST. Plasmid pGEX 4T1™ (Amersham biosciences) was used as template for primers pQEGSTUp and pQEGSTDown (see Table 2), this way the glutathione-S-transferase gene was amplified. The PCR product was gel-purified, BamHI and HindIII digested, and ligated into digested, dephosphorylated pQE-30 vector. This way the 6× Histidine polypeptide and the GST peptide were fused in frame.

His+Bd37-Core+DAF

The DNA fragment encoding the 6× His-Bd37-DAF protein was constructed after three rounds of PCR: in the first round, pBK-CMV-Bd37 plasmid was used as template for primers T3 and Bd37recursUp (see Table 2). The resulting PCR product was gel purified, and used as template for a second round of PCR: 100 ng of the PCR product was amplified with 50 nM of primer Bd37recursEnd and 400 nM each of primers T3 and Bd37DAFc. The resulting PCR product contained the Bd37 core, with its native N-terminal hydrophobic sequence and a C-terminal fusion of the DAF C-terminal hydrophobic region. This PCR product was gel purified, and given a 3′ deoxy-Adenosine overhang by a 30 min incubation with Taq polymerase (Sigma) in amplification buffer comprising 1 mM ATP at 72° C., and was then cloned into plasmid pCR-II (Invitrogen) using the TOPO TA™ cloning kit (Invitrogen). Finally this construct was used as template for primers pQEUp and pQEBd37DAF, to introduce BamHI and HindIII restriction sites, and to remove the N-terminal signal sequence. This fragment was cloned in digested dephosphorylated pQE-vector as described above. The completed construct was verified by DNA-sequencing, performed by Genome Express S.A. (Meylan, France) using the Big Dye terminator method. The resulting insert comprised the Bd37-core fused N-terminally to 6× His and C-terminally to the DAF-C-terminal hydrophobic region.

GST+Bd37-Core

Using similar procedures as outlined above, a plasmid able to express an in frame fusion of GST peptide and Bd37 core polypeptide was constructed. To this purpose pBK-CMV-Bd37 plasmid was used as template for primers pQEUp and pQE70N. These primers provide the resulting amplified fragment with in frame BamHI restriction sites. The PCR product was digested with BamHI and ligated to BamHI digested and dephosphorylated pGEX vector. Ligation product was transfected into JM109 cells, cells were plated, pGEX-GST-Bd37 plasmid was isolated and verified for recombinant protein expression by protein mini-expression method, as described above (except that the lysis buffer was PBS containing lysozyme at 1 mg/ml and 1% v/v TX-100, the washing buffer was the same as the lysis buffer and elution was performed in 50 mM Tris, pH=8 containing 45 mM Glutathione (Sigma).

The various recombinant (rec) proteins that could be expressed from these plasmid inserts are depicted graphically in FIG. 2.

TABLE 2

Primers and linkers used in the
construction of the Bd37 constructs

| | | |
|---|---|---|
| | | SEQ |
| | DNA primer sequence | ID |
| Name | (from 5′to 3′) | NO |

| | | |
|---|---|---|
| T3 | ATTAACCCTCACTAAAGGGA | 1 |
| pQEUp | AATGGCAATAATGGATCCTGCACCAATCTC | 2 |
| pQEDown | GAAGGATGGCTTAAGCTTACTAGATCCCTG | 3 |
| pQEGSTUp | ACACAGGAAACAGGATCCATGTCCCCTATA | 4 |
| pQEGST- | CGCGAGGCAGATAAGCTTTCAGTCACGATG | 5 |
| Down | | |
| Bd37recurs | CGTGTGCCCAGATAGAAGACGGGTAGTACCTGAAGT | 6 |
| Up | ACTAGATCCCTGACCTGATCCTGCAGC | |
| Bd37recurs- | CGTCTTCTATCTGGGCACACGTGTTTCACGTTGACA | 7 |
| End | GGTTTGCTTGGGACGCTAGTAACCATGGGCTTGCTG | |
| | ACTTAG | |
| Bd37DAFc | CTAAGTCAGCAAGCCCATGGTTAC | 8 |
| pQEBd37- | CCCAAGCTTCTAAGTCAGCAAGCCCAT | 9 |
| DAF | | |
| pQE70N | TGGCTTCTTAGGACTGGATCCCTGACCTGA | 10 |
| Bd37HG3′- | CGATTTCGCTGCTGTACCTTCTTCTTTGTCTGCCAT | 11 |
| forw | TGTCTTCGGTATCATTGTATCAATGTTCCG | |
| Bd37HG3′- | GTCCGGAACATTGATACAATGATACCGAAGACAATG | 12 |
| rev | GCAGACAAAGAAGAAGGTACAGCAGCGAAAT | |

### Example 2

### Expression of Rec Proteins

Bacterial Protein Expression:

Bacterial Transfection:

For each recombinant protein to be expressed, an overnight preculture of E. coli was transfected by electroporation. Constructs in pQE plasmids were transfected into E. coli strain M15[pREP4] (Qiagen), and the pGEX-GST-Bd37 plasmid was transfected into E. coli BL 21 (Amersham biosciences). Electroporation was performed using the Prokaryote module of a GenePulser II™ (Bio-Rad), in 1 mm cuvettes (Bio-Rad) in an electroporation medium of 272 mM glucose, 5 mM MgCl₂ and 10% (v/v) glycerol in water. The electric pulse was set to 1.5 kV, 200 Ω, 25 μF. Next the cells were incubated in SOC medium for one hour at 37° C., and plated on ampicilin containing LB agar plates. The next day individual colonies were checked for possession of the correct plasmid by plasmid-miniprep using JetQuick miniprep kit (Q-Bio-Gene), and some of these were tested for correct expression of the desired rec protein by protein mini-expression as described above.

Bacterial Expression:

pQE Constructs:

E. coli M15[pRE4] cells containing the different pQE plasmid constructs were each cultured overnight in LB medium at 37° C. containing 100 μg ampicilin, 25 μg/ml kanamycin, and 0.01% v/v antifoam 209 (Sigma). Next morning the culture was diluted 1:10 in fresh medium and cultured for an additional hour. Then expression of the inserted fragment was induced by addition of 1 mM IPTG. Culturing was continued for4 additional hours. Next cells were pelleted by centrifugation (4000×g, for 20 min) and resuspended in Histag lysis buffer containing 1% v/v Triton X-100™, 1 mg/ml lysosyme and 1 mM phenyl-methyl-sulphonyl fluoride (PMSF) (Sigma). Lysate was stored at −80° C. until use.

After thawing, 500 U DNAse I enzyme (Life Technologies) was added, incubated 20 min on ice, next the suspension was sonicated on ice for 2 minutes with 2 second pulse-pause cycles. The sonicate was centrifuged at 9000×g for 20 minutes. The supernatant was filtered sequentially through 1.2, 0.45 and finally through 0.22 μm filters (Pall Gelman, France). Finally the filtrate was separated on FPLC Ni²⁺ HiTrap™ columns (Pharmacia). The loaded column was washed with Histag lysis buffer supplemented with 20 mM Imidazole (Sigma). Finally the rec proteins were eluted in Histag lysis buffer containing 200 mM Imidazole.

pGex constructs:

E. coli BL21 cells containing the pGEX plasmid constructs were each cultured overnight in LB medium containing 100 μg/ml ampicilin and 0.01% v/v antifoam 209 (Sigma), at 37° C. The culture was diluted 1:10 in fresh medium and culturing was continued for an hour. Protein expression was induced by addition of 0.1 mM IPTG, culturing was continued for 3 additional hours. Cells were pelleted as described above, resuspended in phosphate buffered saline (PBS) containing 1% v/v Triton X-100™, 1 mg/ml lysosyme, and 1 mM PMSF. As-described-above, the lysate was stored at −80° C., thawed, mixed with DNAse I, sonicated and centrifuged. The supernatant was purified over Glutathione-Sepharose beads (Sigma). The beads were washed with PBS/1% X-100™ and rec protein was eluted in a buffer containing 50 mM Tris (pH 8) with 45 mM Glutathione (Sigma).

SPA-Rouen 1987

Soluble parasitic antigen (SPA) represents the culture supernatant of cultures of B. divergens infected erythrocyte cultures. As described by Carcy et al. (1995, Infect. Imm. vol. 63, p. 811-817). Based on computerprediction of the cleavage of the hydrophobic end regions of Bd37, the SPA form comprises a version of the Bd37-ore polypeptide which is slightly larger at the N-terminal side compared to the Bd37-core polypeptide used in the recombinant constructs; it is predicted to consist of Asn-20 up to and including Ser-316 of the Bd37 protein. Additionally, differences in posttranslational processing between the erythrocyte and the bacterial host cells may exist.

Briefly, human erythrocytes were cultured in RPMI 1640 (Invitrogen) and 5 g/l Albumax (purified bovine serum albumin, Invitrogen), at 37° C., in a 5% CO₂ atmosphere. This culture was infected with B. divergens parasites, from French human isolate: Rouen 1987, at an initial parasitemia of 1%, at 5% hematocrite. The culture medium was refreshed daily, until 30-40% parasitemia was reached. At that time the culture medium was used to prepare the control-vaccine with Quil A.

FIG. 3 depicts an SDS-PAGE gel stained with CBB, on which several of the rec proteins are visualized. Gels were run using standard conditions. Briefly: rec protein samples from bacterial cultures as described above, were boiled in sample buffer, loaded onto 12% poly-acrylamide gels and run in Tris-Glycine-SDS running buffer at 140 V, until the bromophenol band contained in the SDS-PAGE sample buffer reached the bottom of the gel. Next the gels were stained and fixed in methanol-acetic acid-CBB and destained overnight. Gels were dried under vacuum for 1 hour at 80 ° C. and finally scanned for storage in digital form.

Because in each lane the same amount of bacterial sample was applied, the relative differences in band intensity reflect the reduced expression efficiency of constructs harboring a hydrophobic peptide. Compare e.g. lanes 2 and 3: 6× His+Bd37-core is expressed much more efficiently than 6× His+Bd37-core+DAF

### Example 3

### Vaccination-Challenge Experiments

Experimental Vaccines

The purified bacterially expressed rec protein was quantitated spectrophoto-metrically along standard samples, using a Coomassie based protein assay kit (Pierce). Then it was diluted in RPMI medium without serum to a final protein concentration of 1 μg in an RPMI volume of 250 μl, and 3 ml of vaccine was prepared (sufficient for 12 gerbils).

For the SPA vaccine, 3 ml of the B. divergens Rouen 1987 Albumax culture medium was used in a similar way.

A fresh stock solution of saponin Quil A, batch L77-163 (Superfos, Denmark) was made at 10 mg/ml in RPMI medium, and 90 μl of this solution was added and mixed by tube flicking (34 time, at room temperature) with the 3 ml vaccine solutions. The final amount of saponin in each 250 μl vaccine dose therefore was 75 _82 g.

Gerbil Immunizations:

Each of the rec protein vaccines was applied to the animals of a group of ten gerbils (Meriones unguiculatus) of 8-9 weeks old, housed in one cage. Animals were marked for individual recognition. Two injections of 250 μl were applied subcutaneously at three-week intervals.

As controls, SPA (culture supernatant of B. divergens Rouen 1987) at 250 μl/dose was included.

One group of gerbils was not vaccinated, and served as challenge control.

At three weeks after the second vaccination a challenge infection was applied, consisting of an intraperitoneal injection of 1000 gerbil red blood cells infected with B. divergens strain Munich. The challenge parasites had previously been passaged through gerbils three times to assure their virulence.

At several times pre- and post-challenge blood samples were taken to monitor the development of anemia and parasitemia. To reduce animal stress, these values were determined of half the number of gerbils at each sampling date, alternating the sub-groups at subsequent dates. Of each experimental group the blood samples of a certain sampling date were pooled. Hematocrit was expressed as % packed cell volume (% PCV) and parasitemia was read microscopically from thin blood smears.

Results and Discussion:

TABLE 3
Results of the vaccination-challenge experiment.

| | Dose | | % Parasitized | % Gerbils with | |
|---|---|---|---|---|---|
| Vaccine-protein | (μg) | | gerbils | ↓ ht > 30%⁽¹⁾ | % Survival |
| His+GST | 1 | | 100 | 100 | 0 |
| His+Bd37-core | 1 | | 100 | 100 | 0 |
| His+Bd37-core+DAF | 1 | | 10 | 10 | 90 |
| GST+Bd37-core | 1 | | 100 | 90 | 20 |
| SPA - Rouen 1987 | 250 | μl | 100 | 70 | 60 |

| | | | | |
|---|---|---|---|---|
| Non-vaccinated | — | 90 | 90 | 10 |

⁽¹⁾Percentage of gerbils per group with a drop in hematocrite larger than 30%.

Except for one animal, all non-vaccinated control animals died after challenge infection, indicating the challenge was of sufficient severity to allow conclusions on vaccine-efficacy. Similar results were obtained for the gerbils immunized with the 6× His+GST, and the 6× His+Bd37-core fusion proteins, in which groups all animals died with total parasitemia and severe anemia (drop in hematocrit value >30%). This indicates that no effective protection can be achieved with the 6× His or the GST peptide components, nor with the Bd37-core polypeptide itself. As expected 6× His does not qualify as heterologous hydrophobic peptide for the invention.

Some protection from challenge was observed in the animals receiving the GST+Bd37-core fusion protein vaccine. Possibly this was due to the size of the fusion protein (GST: 28+Bd37-core: 32 kDa).

However this level of protection was much less than that obtained from the His+Bd37-core+DAF fusion protein vaccine. This fusion protein with hydrophobic C-terminus in Quil A was able to protect all but one of the vaccinated animals from death and even from signs of infection (parasitemia and anemia). This difference in survival rate was statistically significant compared to that of controls (p<0.01, X² test).

As described in EP 1050541, native Bd37 SPA Rouen 1987 in Quil A does protect gerbils to heterologous challenge infection. However, in the present experiment the severity of the heterologous challenge made that only 6/10 animals survived, while 7/10 gerbils developed anemia.

Because the challenge strain used (Munchen) was different from the one used to obtain the Bd37-core polypeptide and the SPA protein (Rouen 1987), it is termed a heterologous challenge. As is well-known in the art, protection to such a heterologous challenge is much harder obtained than to a homologous one.

No significant negative local reactions were observed with the amount of Quil A (75 μg/dose) that was used in these vaccinations.

Conclusion:

These vaccination-challenge experiments show that through the presence of a heterologous hydrophobic sequence fused to the Bd37-core, this fusion protein in Quil A is able to protect 9/10 animals against the consequences of a severe heterologous challenge infection which killed 9/10 unvaccinated animals. Hardly any protection was observed with a vaccine of a control fusion protein or an unfused Bd37-core polypeptide in Quil A. Moderate protection was induced by vaccination with the “native Bd37-core” polypeptide (SPA) in Quil A.

### Example 4

### Construction, Expression, and Use in Vaccination of an AIV H5 Protein with Heterologous Hydrophobic C-Terminal Fusion

Construction of an AIV HA5 Gene with Hydrophobic C-Terminal Fusion:

The HA5 gene from the avian influenza virus (AIV) strain A/chicken/Italy/8/98 (H5N2) was available as a cDNA. This was cloned into the pFastbac1 vector of the baculovirus expression system Bac-2-Bac™ (Invitrogen). Next the construct was digested with ClaI and RsrII restriction enzymes, which removed a fragment comprising the C-terminal 42 amino acids from the HA5 gene, thereby deleting the complete coding region of the transmembrane region of the corresponding H5 protein.

Two linkers: Bd37HG3′-forw, and Bd37HG3′-rev (see Table 2) were designed, that encode the C-terminal 20 amino acids of the Bd37 gene, which is a hydrophobic region with the amino acid sequence: FAAVPSSLSAIVFGIIVSMF. The two linkers also comprised restriction sites: a 5′ ClaI site and a 3′ RsrII site, which form upon annealing of the two linkers.

The two linkers were annealed and ligated into ClaI-RsrII digested pFastBacHA5 plasmid, to construct plasmid pFastBacHA5-Bd37. This HA5-Bd37 construct now encoded AIV H5 protein with a C-terminal fusion of the hydrophobic C-terminus from Bd37 protein. The C-terminal amino acid sequence of this HA5-Bd37 construct is (starting at AIV-H5 amino acid 516): EISGVKLEFAAVPSSLSAIVFGIIVSMF.

Expression in Baculovirus:

Generation of recombinant baculoviruses from plasmid pFastBacHA5 was according to the manufacturer's instructions (Invitrogen). Cells used for expression were Sf9 and Sf158 cells, these were cultured in Bellco microcarrier spinner flasks of 100 and 250 ml. Culture media used were serum free culture media CCM3™ (Hyclone), and SF900-II™ (Invitrogen). Cells were infected at an m.o.i. of 0.1-0.5 and cultured for 3-4 days. Infected insect cells were harvested, and tested via immunofluorescence and Western blotting for presence of H5 protein.

HA5-Bd37 protein-containing insect cells will be purified and H5 protein will be quantified in a standard H5-antigen Elisa. The protein-containing insect cells will be formulated with Quil A™ saponin adjuvant for vaccination of chickens, such that 2 μg HA5-Bd37 and 30 μg Quil A are present per ml of vaccine.

Vaccination of Chickens with Baculovirus Expressed HA5-Bd37:

Chickens will be inoculated with HA5-Bd37 protein-containing insect cells in Quil A and seroconversion will be measured. To this purpose 15 3-week old SPF White leghorn chickens, placed in isolators, will be injected intramuscularly in the leg with 0.25 ml of HA5-Bd37/QuilA vaccine (containing 5 μg HA5-Bd37/dose). At three, four, and five weeks after vaccination blood samples will be drawn. From the clotted blood, the serum will be harvested, inactivated at 56° C., and tested for antibodies to H5, using a standard H5-antibody Elisa.

### LEGEND TO THE FIGURES

FIG. 1: Hydrophobicity profile according to the Kyte and Doolittle hydropathy algorithm. Amino acid numbers are presented along the horizontal axis. The vertical axis lists relative values of hydrophilicity/hydrophobicity; positive values, represented below the median (horizontal axis), correlate to hydrophobic amino acids. Window size was 5 aa. To the right of the graphs are listed the number of hydrophobic data points, and their percentage over the total number of aa in the peptide. The represented peptides (also described in Table 1) are;
Melittin, N-terminus
DAF, C-terminus
CWP 1: Sacharomyces cell wall protein 1, C-terminus
MV HN: measles virus hemagglutinin-neuraminidase, internal region
HHV4 EBNA-3C: human herpesvirus 4 EBNA-3C nuclear antigen, internal region

FIG. 2: Graphical representation of the recombinant proteins employed in the experiments.

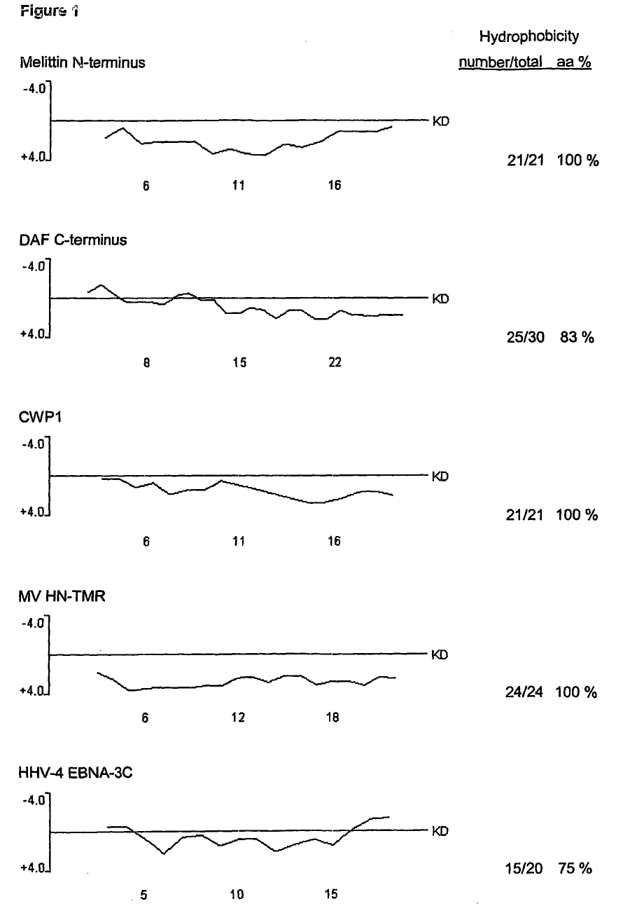

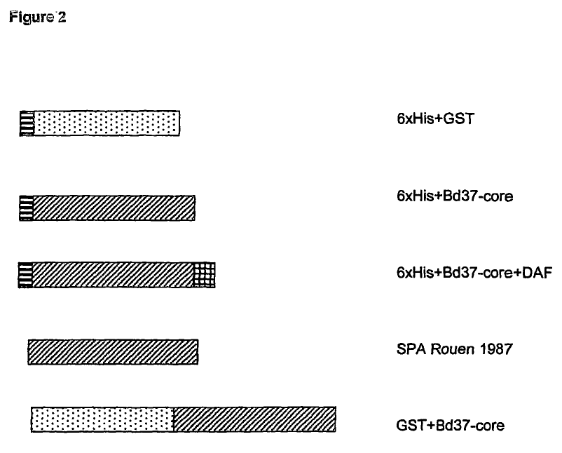

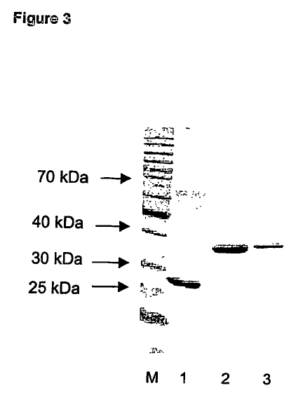

FIG. 3: SDS-PAGE of recBd37 proteins, used for gerbil vaccination; recBd37 proteins were expressed in bacteria, purified using Ni-column chromatography, and loaded onto SDS-PAGE gels. After electrophoresis, the gel was stained, dried and scanned. Lanes are as follows:
M: Molecular weight marker
1: 6× His+GST
2: 6× His+Bd37-core
3: 6× His+Bd37-core+DAF

1. An immunogenic composition comprising a fusion protein and a saponin adjuvant, characterized in that the fusion protein comprises a heterologous hydrophobic peptide which is fused to the N-terminus and/or to the C-terminus of a core polypeptide, the core polypeptide comprising at least one protective epitope, the saponin adjuvant being in a free form.

2. The immunogenic composition according to claim 1, characterized in that the core polypeptide is a component of a protein of an organism of the phylum Apicomplexa.

3. The immunogenic composition according to claim 2, characterized in that the core polypeptide is a component of a protein of an organism of the Piroplasmida or of the class Coccidia.

4. The immunogenic composition according to claim 3, characterized in that the core polypeptide is a component of a protein of an organism of the genera Eimeria or Babesia.

5. The immunogenic composition according to claim 1, characterized in that the heterologous hydrophobic peptide is from an N-terminal hydrophobic sequence.

6. The immunogenic composition according to claim 1 any one of claims 1 to 4, characterized in that the heterologous hydrophobic peptide is from an internal hydrophobic sequence.

7. The immunogenic composition according to claim 1, characterized in that the heterologous hydrophobic peptide is from a C-terminal hydrophobic sequence.

8. The immunogenic composition according to claim 7, characterized in that the C-terminal hydrophobic sequence is from decay accelerating factor (DAF).

9. The immunogenic composition according to claim 1, characterized in that the saponin adjuvant is Quillaja saponin.

10. A vaccine characterized in that it comprises an immunogenic composition according to claim 1 and a pharmaceutically acceptable carrier.

11. The vaccine according to claim 10, characterized in that it comprises at least one additional immunoactive component.

12. The vaccine according claim 10, characterized in that it is in a freeze-dried form.

13. The method for the preparation of a vaccine according to claim 10, characterized in that the method comprises admixing an immunogenic composition according to claim 1 and a pharmaceutically acceptable carrier.

14. (canceled)


SEQUENCE LISTING (12 sequences)

SEQ ID NO: 1 - DNA, 20 bp, Artificial
  Primer, probe, linker etc.
attaaccctcactaaaggga

SEQ ID NO: 2 - DNA, 30 bp, Artificial
  Primer, probe, linker etc.
aatggcaataatggatcctgcaccaatctc

SEQ ID NO: 3 - DNA, 30 bp, Artificial
  Primer, probe, linker etc.
gaaggatggcttaagcttactagatccctg

SEQ ID NO: 4 - DNA, 30 bp, Artificial
  Primer, probe, linker etc.
acacaggaaacaggatccatgtcccctata

SEQ ID NO: 5 - DNA, 30 bp, Artificial
  Primer, probe, linker etc.
cgcgaggcagataagctttcagtcacgatg

SEQ ID NO: 6 - DNA, 63 bp, Artificial
  Primer, probe, linker etc.
cgtgtgcccagatagaagacgggtagtacctgaagtactagatccctgacctgatcctgcagc

SEQ ID NO: 7 - DNA, 78 bp, Artificial
  Primer, probe, linker etc.
cgtcttctatctgggcacacgtgtttcacgttgacaggtttgcttgggacgctagtaaccatgggcttgctgacttag

SEQ ID NO: 8 - DNA, 24 bp, Artificial
  Primer, probe, linker etc.
ctaagtcagcaagcccatggttac

SEQ ID NO: 9 - DNA, 27 bp, Artificial
  Primer, probe, linker etc.
cccaagcttctaagtcagcaagcccat

SEQ ID NO: 10 - DNA, 30 bp, Artificial
  Primer, probe, linker etc.
tggcttcttaggactggatccctgacctga

SEQ ID NO: 11 - DNA, 66 bp, Artificial
  Primer, probe, linker etc.
cgatttcgctgctgtaccttcttctttgtctgccattgtcttcggtatcattgtatcaatgttccg

SEQ ID NO: 12 - DNA, 67 bp, Artificial
  Primer, probe, linker etc.
gtccggaacattgatacaatgataccgaagacaatggcagacaaagaagaaggtacagcagcgaaat